(19) United States
(12) Patent Application Publication
(10) Pub. No.: US 2014/0364368 A1
(43) Pub. Date: Dec. 11, 2014

(54) STIMULUS RESPONSIVE NANOCOMPLEXES AND METHODS OF USE THEREOF

(71) Applicant: MASSACHUSETTS INSTITUTE OF TECHNOLOGY, Cambridge, MA (US)

(72) Inventors: Kevin Yu-ming Lin, Cambridge, MA (US); Sangeeta N. Bhatia, Lexington, MA (US); Gabriel Abner Kwong, Boston, MA (US); Justin Han-je Lo, Cambridge, MA (US)

(21) Appl. No.: 14/298,317

(22) Filed: Jun. 6, 2014

Related U.S. Application Data
(60) Provisional application No. 61/831,916, filed on Jun. 6, 2013.

(51) Int. Cl. A61K 47/48; A61K 31/715

(52) U.S. Cl. CPC A61K 47/48246; A61K 31/715
USPC 514/14.9; 530/326

(57) ABSTRACT

The present invention provides stimulus responsive nanocomplexes comprising a masking moiety, e.g., a peptide, and a therapeutic moiety, e.g., an anti-coagulant. The invention also provides methods for treating or preventing a condition, such as a hypercoagulable state, e.g., blood clotting disorders or a cardiovascular disease, in a subject.

### RELATED APPLICATIONS

This application claims the benefit of U.S. Provisional Application No. 61/831,916, filed on Jun. 6, 2013. The entire contents of the aforementioned priority application are hereby incorporated herein by reference.

### GOVERNMENT SUPPORT

This invention was made with government support under grant number 1-R01-CA124427, awarded by the National Institutes of Health. The government has certain rights in the invention.

### BACKGROUND OF THE INVENTION

Homeostatic regulation pervades diverse processes which play critical roles in human health, including hormone release, ionic balance, and cell-mediated immunity. In particular, the body employs negative feedback loops to keep these processes within physiologic limits while preventing runaway amplification cascades or positive feedback cycles. A key example of a homeostatically-regulated process with significant medical relevance is blood coagulation, the protease-driven positive-feedback cascade by which clots are formed to stop blood loss from a damaged vessel. Dysregulation of this process, whether pathological or drug-induced, leads to two adverse outcomes: too little coagulation may lead to life-threatening hemorrhage and hypovolemic shock, while overactive coagulation may lead to thrombosis (clotting within the blood vessel), the potentially fatal medical condition underlying pulmonary embolism, stroke, and organ infarction.

An essential step in the coagulation, or blood clotting, cascade is the proteolytic cleavage of fibrinogen to release fibrinopeptides A and B. These peptides, in turn, lead to the generation of fibrin which can undergo polymerisation to form a hemostatic plug, or ‘blood clot’. Although the blood coagulation cascade may be modulated at numerous different sites, a rate limiting step in this process is the cleavage of fibrinogen, which is catalyzed by the trypsin-like serine protease thrombin. Anti-coagulants are pharmacological agents inhibit thrombin activity, thereby preventing the action of thrombin in the blood coagulation cascade. The most common side effect of anti-coagulants is the occurrence of hemorrhagic complications, which can on occasion prove fatal.

The need for tight control of coagulation explains the narrow therapeutic windows of anti-coagulants, even when they are administered with strict dose titration and monitoring. One example of such anti-coagulant is unfractionated heparin (UFH), a mainstay in the hospital setting, which is particularly difficult to dose properly because of its unpredictable pharmacokinetics. Clotting time measurements and dose re-adjustment may be required up to every 3-4 hours to maintain UFH levels within the therapeutic range (Hirsh J. et al., Chest (2001), 119:64S-94S).

For the past century, pharmaceutical development of anti-coagulants has focused on targeting novel molecular entities within the coagulation cascade, yet regardless of the target, these drugs globally inhibit clot formation and inherently increase the risk of bleeding (Mannucci P. et al., Annals of Medicine (2011), 43:116-123; Melnikova I., Nature Reviews Drug Discovery (2009), 8:353-4). Several examples of engineering approaches to systemic anti-coagulant delivery have been demonstrated in the literature to date, although none directly addresses the serious bleeding side-effects. Methods utilizing nanoparticle delivery have focused on improving the drug efficacy through multivalent interactions or by targeting anti-coagulant particles to locations prone to thrombus formation, but still lack active mechanisms to limit bleeding (Peters et al., PNAS (2009), 106:9815-9819; Shiang et al., Angew. Chemie (2011), 50:7660-7665). Polymer microsphere formulations have been tested as oral delivery vehicles for heparin, which has low gastrointestinal absorption, but do not alter the bleeding profile of the drug itself (Jiao et al., J. Pharm. Sci. (2002), 91:760-768); Jiao et. al., Circulation (2002), 105:230-235). Other strategies have explored the use of biodegradable polymers for controlled release of anticoagulants (Vasudev et al., Biomaterials (1997), 18:375-81; Gutowska et al., J. Biomed. Mater. Res. (1995), 29:811-21; Baldwin et al., J. Biomed. Mater. Res. (2012), 100A:2106-18). The aforementioned approaches all rely on the classic paradigm of passive delivery mechanisms that are wholly dissociated from the disease state and therefore not designed to mitigate the current bleeding risks of anticoagulation. Further, such open-loop systems deliver their cargo without any form of feedback regulation and cannot autonomously titrate the release of drugs in response to the dynamic circulatory conditions within the body.

An alternative strategy to wholesale, unrestricted anticoagulation employed engineering of an active mechanism for releasing an anti-coagulant at the time and site of a thrombotic event in order to maximize therapeutic efficacy while offsetting bleeding risk. Similar strategies have been applied to the engineering of bioresponsive thrombolytics, another arm of antithrombotic therapy designed to dissolve existing clots where therapeutic activation is initiated by a proteolytic or biophysical triggers associated with thrombosis. Previously, a recombinant thrombin-activated variant of human plasminogen was introduced that demonstrated selective generation of plasmin, a component of the anti-clot cascade, localized to newly formed clots without affecting established clots or bleeding time (Dawson et al., J. of Biol. Chem. (1994), 269:15989-92; corner et al., J. of Thrombosis and Haemostasis (2005), 3:146-153). This technology has since entered testing in clinical trials (Curtis et al., J. of Thrombosis and Haemostasis (2005), 3:1180-1186; Gibson et al., J. of Thrombosis and Thrombolysis (2006), 22:13-21). Recently, shear-activated microparticles were designed that released tissue plasminogen activator (tPA) only in thrombosed vessels upon exposure to local shear stresses one to two orders of magnitude higher than those present in normal vasculature (Korin et al., Science (2012), 337:738-742). This technology required lower doses of the drug and produced fewer side-effects than treatment with free tPA in mouse models of embolism.

Although the thrombin-activated plasminogen and the shear-activated microparticles represent bioresponsive thrombolytics that may be used to dissolve already existing clots, there still exists a need in the art for more efficacious bioresponsive anticoagulants that can be used prophylactically to prevent the formation of future clots. There also exists a need in the art for anticoagulants that would have predictable dosing profiles and reduced side effects.

### SUMMARY OF THE INVENTION

The present invention provides stimulus responsive nanocomplexes comprising a therapeutic agent and a masking moiety. The masking moiety prevents the therapeutic agent from exerting its biological activity and also comprises a sensor responsive to a stimulus. When the sensor is modified in the presence of the stimulus, the masking moiety is no longer able to prevent the therapeutic agent from exerting its biological activity. The present invention also provides methods for treating subjects in need thereof, using these nanocomplexes, e.g., treating subjects suffering from or prone to hypercoagulable states.

Accordingly, in one aspect, the present invention provides a stimulus responsive nanocomplex. The nanocomplex includes a therapeutic agent; and a masking moiety comprising a sensor responsive to a stimulus, wherein the masking moiety prevents the therapeutic agent from exerting its biological activity, and wherein the sensor is modified in the presence of the stimulus, thereby allowing the therapeutic agent to exert its biological activity. In some embodiments, the masking moiety comprises a proteinaceous compound. In some embodiments, the proteinaceous compound comprises a peptide.

In certain embodiments, the sensor comprises a protease sensitive sequence. In one embodiment, the protease sensitive sequence is a thrombin cleavage sequence.

In some embodiments, the stimulus is an agent capable of cleaving the protease sensitive sequence. In some embodiments, the agent capable of cleaving the protease sensitive sequence is a clot-forming agent. In a further embodiment, the clot-forming agent is a protease. In one specific embodiment, the protease is thrombin.

In some embodiments, the therapeutic agent is a blood homeostasis agent. In certain embodiments, the blood homeostasis agent is an anti-coagulant. In specific embodiments, the anti-coagulant is heparin or bivalirudin.

In some embodiments, the nanocomplex is self-assembling. In other embodiments, the nanocomplex is self-titrating.

In certain embodiments, the therapeutic agent and the masking moiety interact directly with each other to form the nanocomplex.

In some embodiments, the therapeutic agent is a charged therapeutic agent, and the masking moiety is a charged moiety. In certain embodiments, the charged therapeutic agent is negatively charged and the charged masking moiety is positively charged. In other embodiments, the charged therapeutic agent is positively charged, and the charged masking moiety is negatively charged.

In some embodiments, the masking moiety is a peptide. In certain embodiments, the therapeutic agent is a blood homeostasis agent. In some aspects, the blood homeostasis agent is an anti-coagulant. In one specific embodiment, the anti-coagulant is heparin.

In certain aspects, the sensor comprises a protease sensitive sequence. In a specific embodiment, the protease sensitive sequence is a thrombin cleavage sequence.

In some embodiments, the therapeutic agent and the masking moiety interact indirectly with each other. In some embodiments, the stimulus responsive nanocomplex further comprises a nanoparticle. In a specific embodiment, the nanoparticle is an iron oxide nanoparticle.

In some embodiments, the therapeutic agent and the masking moiety both interact with the nanoparticle. In one embodiment, the masking moiety is a peptide.

In certain embodiments, the therapeutic agent is a blood homeostasis agent. In some embodiments, the blood homeostasis agent is an anti-coagulant. In a specific embodiment, the anti-coagulant is bivalirudin.

In some embodiments, the sensor comprises a protease sensitive sequence. In one embodiment, the protease sensitive sequence is a thrombin cleavage sequence.

In certain embodiments, the masking moiety in the stimulus responsive nanocomplex comprises a peptide. In some embodiments, the peptide is further conjugated to a polymeric agent. In one specific embodiment, the polymeric agent is polyethylene glycol (PEG).

In another aspect, the present invention provides a stimulus responsive nanocomplex, including a charged therapeutic agent; and a charged peptide comprising a sensor responsive to a stimulus, wherein the charge of the peptide is opposite to the charge of the charged therapeutic agent thereby allowing the formation of the nanocomplex and wherein in the presence of the stimulus the sensor is modified thereby releasing the charged therapeutic agent from the nanocomplex. In some embodiments, the charged therapeutic agent is negatively charged and the charged peptide is positively charged. In other embodiments, the charged therapeutic agent is positively charged and the charged peptide is negatively charged.

In certain embodiments, the charged therapeutic agent is a blood homeostasis agent. In some embodiments, the blood homeostasis agent is an anti-coagulant. In one specific embodiment, the anti-coagulant is heparin.

In some embodiments, the sensor is a protease sensitive sequence. In a specific embodiment, the protease sensitive sequence is a thrombin cleavage sequence.

In certain aspects, the stimulus is an agent capable of cleaving the protease sensitive sequence. In some embodiments, the agent is a clot-forming agent. In certain embodiments, the clot-forming agent is a protease. In one specific embodiment, the protease is thrombin.

In some embodiments, the charged peptide masks the biological activity of the charged therapeutic agent.

In yet another aspect, the present invention provides a nanocomplex for the treatment or prevention of thrombosis, the nanocomplex including a negatively charged anti-thrombotic agent; and a positively charged peptide, wherein the peptide comprises a protease sensitive sequence.

In one embodiment, the anti-thrombotic agent is heparin. In some embodiments, the peptide is in excess of the anti-thrombotic agent within the nanocomplex.

In some embodiments, the nanocomplex is formed by the self-assembly of the anti-thrombotic agent and the peptide. In further embodiments, the peptide masks the negative charge of the anti-thrombotic agent. In one embodiment, the protease sensitive sequence is a thrombin cleavage sequence.

In a further aspect, embodiment, the present invention provides a nanocomplex for the treatment or prevention of thrombosis, the nanocomplex including heparin; and a positively charged peptide comprising a thrombin cleavable sequence.

In some embodiments, the peptide is in excess of the heparin within the nanocomplex. In some embodiments, the nanocomplex is self-assembling. In certain embodiments, the peptide masks the negative charge of the heparin.

In another aspect, the present invention provides a nanocomplex for the treatment or prevention of thrombosis, the nanocomplex including bivalirudin; a peptide-polyethylene glycol conjugate comprising a thrombin cleavable sequence; and a nanoparticle, wherein both the bivalirudin and the peptide-polyethylene conjugate are attached to the nanoparticle.

In some embodiments, the present invention also provides a method of treating a subject in need thereof, the method comprising administering to the subject an effective amount of the nanocomplex of the invention, thereby treating the subject.

In some embodiments, the subject is suffering from a hypercoagulable state. In certain embodiments, the hypercoagulable state is hypertension or cardiovascular disease. In some aspects, the cardiovascular disease is coronary occlusion, arteriosclerotic heart disease (ASHD) or coronary thrombosis.

In one embodiment, the subject is a human.

The present invention is further illustrated by the following detailed description and drawings.

### BRIEF DESCRIPTION OF THE DRAWINGS

FIG. 1A is a schematic showing self-assembly of cationic PEG-Peptide and anionic heparin to form nanocomplexes.

FIG. 1B is a schematic illustrating over-anticoagulation leading to increased bleeding.

FIG. 1C is a schematic illustrating negative feedback system for self-titrating release of heparin based on thrombin activity.

FIG. 1D is a schematic illustrating that decreased anticoagulation may not mitigate the risk of thrombosis.

FIG. 2A is a transmission electron microscopy image of nanocomplexes with a 25:5:1 PEG:peptide:heparin molar ratio (scale bar of 100 nm).

FIG. 2B is a graph showing the average hydrodynamic diameter of nanocomplexes at varying PEG:peptide:heparin ratios in PBS and 10% serum (n=3 per condition, s.d.).

FIG. 2C is a graph showing the zeta potential of nanocomplexes at varying PEG:peptide:heparin ratios in PBS and 10% serum (n=3 per condition, s.d.).

FIG. 3 is a graph showing the percent viability of HUVEC cells incubated with varying concentrations of nanocomplexes, PEG-peptide, peptide only and free heparin, as determined by a cytotoxicity MTS assay.

FIGS. 4A-4C are a panel of chromatograms showing absorbance of Superdex 200 column effluent after application of samples containing (A) nanocomplexes (NP), (B) nanocomplexes(NP) incubated with thrombin, and (C) free heparin.

FIG. 5A is a standard curve of free heparin with Azure II (slope=0.037, y-intercept=0.097, r2=0.98; n=3 per condition, s.d.).

FIG. 5B is a graph showing the amount of free heparin (U/mL) released from nanocomplexes incubated in the absence or presence of thrombin as measured using Azure II. Absorbance was compared to the standard curve of free heparin from (A) to determine the amount of free heparin (**P<0.05 by Student's t-test; n=3 per condition, s.d.).

FIG. 6A is a graph showing effective heparin activity as determined by an anti-FXa assay upon complexation with peptides (Pep.) and treatment with thrombin (Thr.). Here, D, refers to the D-isomer.

FIG. 6B is a graph showing the % of heparin released from nanocomplexes as a function of thrombin concentration and incubation time with thrombin. The amount of heparin released was determined using an anti-FXa assay (n=3 per condition, s.d.)

FIG. 6C is a graph showing activated partial thromboplastin time of normal human control plasma spiked with free heparin or nanocomplexes (designated here as “nS”) (**P<0.01; ***P<0.001 by two-way ANOVA with Bonferroni post test; n=3 per condition, s.d.).

FIG. 7A is a graph showing the amount of circulating nanocomplexes (NP) and free heparin (Hep) as measured by an anti-FXa assay on mouse plasma samples over time.

FIG. 7B is a graph showing the amount of circulating nanocomplexes and free heparin as determined by fluorescence using FITC-heparin.

FIG. 7C is a graph showing tail bleeding time for mice that were administered 200 U/kg of heparin or nanocomplexes (**P<0.01 by one-way ANOVA with Tukey post test; n=5-7 mice, s.e.).

FIG. 8 is an ex vivo near-infrared image of mouse organs following co-injection of VT750-fibrinogen and thromboplastin.

FIG. 9 is a graph showing the amount of VT750-fibrin deposition in mouse lungs following co-injection of thromboplastin and escalating doses of heparin (Hep) (n=3-5 mice, s.e.).

FIG. 10A is a graph showing the average fibrin deposition in the lungs of mice dosed with thromboplastin (T, 2 μL/g body weight) and nanocomplexes (NP, 200 U/kg) or free heparin (Hep, 200 U/kg). (**P<0.01 by one-way ANOVA with Tukey post test; n=5 mice, s.e.)

FIG. 10B is an ex vivo near-infrared fluorescent imaging of VT750-fibrin in the lungs of mice administered PBS (1), thromboplastin only (2), thromboplastin+heparin (3) and thromboplastin+nanocomplexes (4).

FIG. 10C is an image of hematoxylin and eosin staining of the lungs in mice administered PBS (1), thromboplastin (2), thromboplastin+heparin (3) and thromboplastin+nanocomplexes (4) under the same conditions as in (B). Arrow denotes fibrin clots; arrowheads denote patent vessels (scale bar=100 μm).

FIG. 11A is a graph showing the average the real-time thrombin generation in human plasma with increasing concentrations of free heparin (Hep) (n=3 per condition).

FIG. 11B is a graph showing the average the real-time thrombin generation in human plasma with increasing concentrations of nanocomplexes (designated here as “nS”) (n=3 per condition).

FIG. 11C is a graph showing the fluorescence signal from the thrombin generation assay. The fluorescence traces result from the cleavage of a fluorogenic substrate by thrombin in normal human control plasma spiked with 0.4 U/mL of nanocomplexes (designated here as “nS”) and is plotted against representative traces from human plasma spiked with various concentrations of free heparin (n=3 per condition; excitation: 360 nm; emission: 460 nm).

FIG. 11D is a graph showing the fluorescence signal from the thrombin generation assay. The fluorescence traces result from the cleavage of a fluorogenic substrate by thrombin in normal human control plasma spiked with 0.6 U/mL of nanocomplexes (designated here as “nS”) and is plotted against representative traces from human plasma spiked with various concentrations of free heparin (n=3 per condition; excitation: 360 nm; emission: 460 nm).

FIG. 11E is a graph showing the average the lag time calculated from the real-time thrombin generation assays in FIGS. 11A and 11B (*P<0.05; **P<0.01; ***P<0.001 by two-way ANOVA with Bonferroni post test; n=3 per condition, s.d.; Hep (); nanocomplexes (designated here as “nS”) (▪)).

FIG. 11F is a graph showing the average the peak thrombin calculated from the real-time thrombin generation assays in FIGS. 11A and 11B (*P<0.05; **P<0.01; ***P<0.001 by two-way ANOVA with Bonferroni post test; n=3 per condition, s.d.; Hep (); nanocomplexes (▪)).

FIG. 11G is a graph showing the average the endogenous thrombin potential (ETP) calculated from the real-time thrombin generation assays in FIGS. 11A and 11B (*P<0.05; **P<0.01; ***P<0.001 by two-way ANOVA with Bonferroni post test; n=3 per condition, s.d.; Hep (▪); nanocomplexes ()).

### DETAILED DESCRIPTION OF THE INVENTION

The present invention provides stimulus responsive nanocomplexes comprising a therapeutic agent and a masking moiety. In the absence of the stimulus, the masking moiety interacts directly or indirectly with the therapeutic agent and prevents the therapeutic agent from exerting its biological activity. When the stimulus is present, the masking moiety no longer masks the therapeutic agent, thereby allowing the therapeutic agent to exert its biological activity.

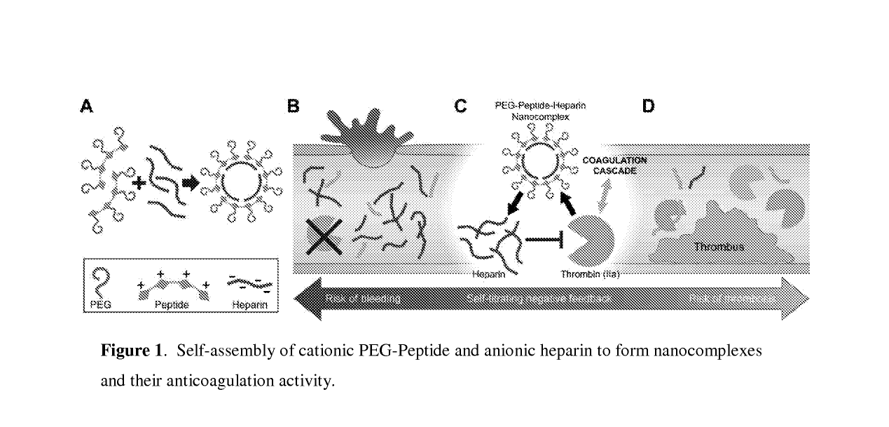

In some embodiments, the therapeutic agent may be a blood homeostasis agent, e.g., an anti-coagulant. In such exemplary embodiments, the benefits provided by the nanocomplexes of the present invention include selective activation of the exogenous anti-coagulant only in response to inappropriate thrombotic events, while smaller-scale clotting in response to everyday compromises of the endothelium, such as bruising or cuts, is not affected. In certain embodiments, the nanocomplexes comprise heparin, an anionic anti-coagulant, that forms charge-based complexes with a cationic thrombin-sensitive peptide (FIG. 1A). The thrombin-activated release mechanism enables heparin delivery localized to sites of thrombin formation and proportional to the amount of thrombin activity, resulting in a formulation that deploys more anti-coagulant under thrombotic conditions, yet is more tolerant of healthy coagulation processes (FIGS. 1B-D). This novel self-titrating anti-coagulant has the potential for equal therapeutic efficacy with fewer bleeding side effects than the unfractionated heparin. Coupled with its decreased bleeding risk, this nanoparticle system of veiled, context-activatable anti-coagulant, e.g., heparin, represents an improved therapy over unfractionated heparin (UFH) by providing an expanded therapeutic window. As a potential direct replacement for UFH in the hospital setting, these self-titrating nanocomplexes may obviate the need for frequent dose monitoring and readjustment, and decrease the risk of and costs associated with bleeding complications, while still retaining the benefits of short circulation time and availability of an antidote if needed in an emergency scenario. Sequestration of a therapeutic agent, such as heparin, in a complex form, may reduce side effects associated with nonspecific binding of the therapeutic agent, e.g., heparin, to endogenous proteins and surfaces, such as dose-dependent mechanisms of clearance and heparin-induced thrombocytopenia.

Furthermore, because the underlying chemistry used to produce the nanocomplexes is very well established, the manufacture of the nanoparticles may be cheaper and simpler, as compared to the other agents known in the art, such as thrombin-activated plasminogen and the shear-activated microparticles.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

As used herein, the term “stimulus responsive nanocomplex” is a complex that comprises a therapeutic agent which becomes available to exert its biological activity in response to a stimulus. In some embodiments, the stimulus responsive nanocomplex comprises a therapeutic agent and a masking moiety. The masking moiety comprises a sensor responsive to a stimulus. In the absence of the stimulus, the masking moiety prevents the therapeutic agent from exerting its biological activity. In the presence of the stimulus, the sensor is modified, thereby allowing the therapeutic agent to exert its biological activity. The therapeutic agent and the masking moiety may interact directly to form the nanocomplex or they may interact indirectly, e.g., through a nanoparticle, to form the nanocomplex. Thus, in some embodiments, the stimulus responsive nanocomplex further comprises a nanoparticle, e.g., an iron oxide nanoparticle. In one embodiment, the stimulus responsive nanocomplex is thrombin-activatable plasminogen or a thrombin-activatable plasminogen analog.

As used herein, the term “therapeutic agent” includes any biologically active agent that may be used in the nanocomplexes of the present invention. In some embodiments, the therapeutic agent may be a small molecule, a peptide, an oligosaccharide, an oligonucleotide, or a protein, e.g., an antibody or a fragment thereof. The therapeutic agent may carry an overall negative charge, a positive charge or may be neutral at physiological conditions. In some embodiments, the therapeutic agent is a blood homeostasis agent, e.g., an anti-coagulant, useful in treating a hypercoagulable state. Specific examples of such agents include, but are not limited to, acenocoumarol (Sinthrome®), apixaban, aspirin, bivalirudin (Angiox®), clopidogrel (Plavix®), fondaparinux sodium (Arixtra®), low molecular weight heparins (e.g., semuloparin, bemiparin, dalteparin, and enoxaparin), heparin, heparin sodium, dabigatran etexilate mesylate (Pradaxa®), danaparoid sodium (Orgaran), epoprostenol sodium (Flolan®), tinzaparin sodium (Innohep®), warfarin (Marevan®), menadiol sodium phosphate, rivaroxaban (BAY 59-7939, Xarelto®), prasugrel, ticagrelor, ticlopidine, argatroban, lepidurin, anagrelide, apixaban, cilostazol, and dipyridamole. In one specific embodiment, the therapeutic agent is heparin. In another specific embodiment, the therapeutic agent is bivalirudin.

As used herein, the term “masking moiety” includes any moiety that, when present as a part of a nanocomplex of the invention, prevents the therapeutic agent from exerting its biological activity, e.g., an anti-coagulant activity. In some embodiments, the masking moiety may interact directly with the therapeutic agent via any type of interaction known in the art. For example, the masking moiety may interact with the therapeutic agent via electrostatic interactions, hydrogen bonding interactions, covalent interactions, Van der Waals interactions or hydrophobic interactions. In one embodiment, both the masking moiety and the therapeutic agent are charged, and interact with each other directly via electrostatic interactions.

In other embodiments, the masking moiety interacts indirectly with the therapeutic agent. In one such embodiment, both the masking moiety and the therapeutic agent are conjugated to a nanoparticle.

The masking moiety may be any entity, such as a small molecule, a peptide, a polypeptide, an oligosaccharide, an oligonucleotide, a peptide nucleic acid (PNA), or a protein, e.g., an antibody or a fragment thereof. In some embodiments, the masking moiety is a peptide, such as a charged peptide, e.g., a positively or a negatively charged peptide. In one specific embodiment, the peptide is positively charged.

In one embodiment, the masking moiety is not a higher ordered structure. In a further embodiment, the masking moiety is not an encapsulating particle or a protein cage, i.e., a structure with an interior cavity which is either naturally accessible to a solvent or can be made to be so by altering solvent concentration, pH or equilibria ratios. In a specific embodiment, the masking moiety is not a virion protein cage. In yet another embodiment, the masking moiety is not, and the nanocomplex does not comprise, a transmembrane polypeptide that naturally comprises a pore (i.e., a channel).

The masking moiety comprises a sensor responsive to a stimulus. The “sensor responsive to a stimulus”, as used herein, is any molecular entity that is modified in response to a stimulus, thereby allowing the therapeutic agent to exert its biological activity. For example, the sensor may undergo a conformation change, a cleavage, a binding, or a degradation in response to a stimulus. In one embodiment, the sensor comprises a protease sensitive sequence, e.g., a thrombin cleavage sequence, and is cleaved in response to exposure to the stimulus, e.g., thrombin.

“Biological activity”, as used herein, is well known in the art and includes any activity by a therapeutic agent, as described herein, that elicits a response from living tissue or an organism. In some embodiments, the biological activity includes any activity exerted by a therapeutic agent comprised in the nanocomplexes as described herein. In a specific embodiment, the biological activity is an anti-coagulant activity that prevents, reduces or inhibits blood clotting.

A “stimulus”, as used herein, includes any set of conditions that produce a change in the sensor. For example, a stimulus may be a specific pH, a specific temperature or a change in temperature, or an agent capable of interacting with the sensor and present in a location at which the activity of the therapeutic agent is needed and/or desired. In some embodiments, the stimulus is an agent capable of cleaving a protease sensitive sequence. In further embodiments, the stimulus is a clot forming agent, e.g., a protease, such as thrombin.

A “hypercoagulable state”, or “thrombophilia”, as used herein, refers to any blood clotting disorder that is characterized by excessive coagulation or any other condition associated with excessive coagulation. In one embodiment, the conditions associated with excessive coagulation include, but are not limited to, any condition characterized by an increased risk of myocardial infarction, pulmonary embolism or a stroke, such as hypertension or cardiovascular disease, e.g., coronary occlusion, arteriosclerotic heart disease (ASHD) or coronary thrombosis.

A hypercoagulable state may be a genetic (inherited) or an acquired condition. Examples of genetic hypercoagulable states include, but are not limited to, Factor V Leiden; conditions caused by prothrombin gene mutation; deficiencies of natural proteins that prevent clotting (such as antithrombin, protein C and protein S); conditions characterized by elevated levels of homocysteine, elevated levels of fibrinogen or by dysfunctional fibrinogen (dysfibrinogenemia); conditions characterized by elevated levels of factor VIII and other factors including factor IX and XI; conditions characterized by abnormal fibrinolytic system, including hypoplasminogenemia, dysplasminogenemia, and elevation in levels of plasminogen activator inhibitor (PAI-1). Acquired hypercoagulable conditions may include, but are not limited to, cancer and associated conditions caused by some medications used to treat cancer, such as tamoxifen, bevacizumab, thalidomide and lenalidomide; recent trauma or surgery; central venous catheter placement; obesity; pregnancy; conditions caused by supplemental estrogen use, including oral contraceptive pills (birth control pills); conditions characterized by hormone replacement therapy; conditions characterized by prolonged bed rest or immobility; heart attack, congestive heart failure, stroke and other illnesses that lead to decreased activity; heparin-induced thrombocytopenia (decreased platelets in the blood due to heparin or low molecular weight heparin preparations); conditions caused by lengthy airplane travel, also known as “economy class syndrome”; antiphospholipid antibody syndrome; previous history of deep vein thrombosis or pulmonary embolism; myeloproliferative disorders such as polycythemia vera or essential thrombocytosis; paroxysmal nocturnal hemoglobinuria; inflammatory bowel syndrome; HIV/AIDS; and nephrotic syndrome characterized by excessive protein in the urine).

### II. Nanocomplexes of the Invention

The present invention provides stimulus responsive nanocomplexes comprising a therapeutic agent and a masking moiety that prevents the therapeutic agent from exerting its biological activity. The masking moiety comprises a sensor responsive to a stimulus. The sensor is modified in the presence of the stimulus, such that the masking moiety no longer prevents the therapeutic agent from exerting its biological activity.

The therapeutic agent may be any biologically active agent that may be used in the nanocomplexes of the present invention. In some embodiments, the therapeutic agent is a blood homeostasis agent, e.g., an anti-coagulant, such as heparin or bivalirudin. In some embodiments, the therapeutic agent may be a charged therapeutic agent, e.g., a positively charged therapeutic agent or a negatively charged therapeutic agent, or a neutral therapeutic agent. In a specific embodiment, the therapeutic agent is a negatively charged therapeutic agent, e.g., heparin. In another specific embodiment, the therapeutic agent is a therapeutic agent with no charge, e.g., bivalirudin.

The masking moiety may be any moiety that, when present in a nanocomplex of the invention, prevents the therapeutic agent from exerting its biological activity. The masking moiety may be any entity, e.g., a small molecule, a peptide, a polypeptide, an oligosaccharide, an oligonucleotide, a peptide nucleic acid (PNA), or a protein, e.g., an antibody or a fragment thereof. In one embodiment, the masking moiety is a peptide. In a further embodiment, the masking moiety is a charged peptide, e.g., a positively or a negatively charged peptide. The positively charged peptide may comprise positively charged amino acids, e.g., arginine or lysine. In a specific embodiment, a positively charged peptide may comprise stretches of alternating arginines and lysines.

In some embodiments, the masking moiety and the therapeutic agent are present in the nanocomplex at a ratio of between about 1:1 to about 10:1 masking moiety:therapeutic agent, e.g., about 1:1, about 1.5:1, about 2:1, about 2.5 to 1, about 3:1, about 3.5:1, about 4:1, about 4.5:1, about 5:1, about 5.5:1, about 6:1, about 6.5:1, about 7:1, about 7:5:1, about 8:1, about 8.5:1, about 9:1, about 9.5:1, or about 10:1. In a specific embodiment, the masking moiety, e.g., a peptide, such as a positively charged peptide, and the therapeutic agent, e.g., a negatively charged therapeutic agent, such as heparin, are present at a ratio of 5:1.

In some embodiments, the masking moiety, e.g., a peptide, may be further conjugated to an additional agent, e.g., a polymeric agent. Conjugation of the masking moiety, e.g., a peptide, to the additional agent, e.g., a polymeric agent may be desirable to prevent the instability and aggregation of nanocomplexes in physiological solutions and at high concentrations. The instability and aggregation of nanocomplexes can negatively impact in vivo performance by decreasing circulation time and increasing risk of lung entrapment.

The polymeric agent may comprise any number of hydrophilic non-fouling polymers. Examples of such polymers include, but are not limited to, polyethylene glycols (PEGs), polyoxazolines, poly(amino acids), N-(2-hydroxylpropyl)methacrylamide (HPMA), polybetaines, polyglycerols, polysaccharides (e.g., hyaluronic acid, dextran and chitosan), and polypeptides.

In some embodiments, the polymeric agent is a member of a family of polyethylene glycols (PEGs). Polyethylene glycols are a family of polymers produced from the condensation of ethylene glycol, and have the general formula H(OCH₂CH₂)ₙOH where n, the number of ethylene glycol groups, is greater than or equal to 4. Generally, the designation of a polyethylene glycol (PEG) includes a number that corresponds to its average molecular weight. For example, polyethylene glycol 1500 refers to a mixture of polyethylene glycols having an average value of n between 29 and 36 and a molecular weight range of 1300 to 1600 grams/mole. In a specific embodiment, the PEG has an average molecular weight of 5000 grams/mole. PEGs may further be covalently linked to additional functional groups, e.g., groups that may allow the PEGs to be linked to other moieties, e.g., a therapeutic agent. In a specific embodiment, the PEG is a poly(ethylene glycol)-succinimidyl valerate.

In some embodiments, the additional agent, e.g., a polymeric agent such as PEG, and the masking moiety, e.g., a peptide are present in the nanocomplex at a ratio of between about 1:1 to about 25:1 additional agent:masking moiety, e.g., about 1:1, about 2:1, about 3:1, about 4 to 1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1 or about 25:1. In a specific embodiment, the additional agent, e.g., a polymeric agent such as PEG, and the masking moiety, e.g., a peptide, are present in the nanocomplex at a ratio of 25:1. In another specific embodiment, the additional agent, e.g., a polymeric agent such as PEG, and the masking moiety, e.g., a peptide, are present in the nanocomplex at a ratio of 5:1.

The masking moiety may interact directly or indirectly with the therapeutic agent. Direct interactions of the masking moiety with the therapeutic agent may involve electrostatic interactions, hydrogen bonding interactions, covalent interactions, Van der Waals interactions or hydrophobic interactions. In some embodiments, where both the masking moiety and the therapeutic agent are charged, e.g., the masking moiety is negatively charged and the therapeutic agent is positively charged, or the masking moiety is positively charged and the therapeutic agent is negatively charged, the interactions between the masking moiety and the therapeutic agent are electrostatic interactions. In a specific embodiment, the masking moiety is a positively charged peptide, e.g., a peptide having the sequence comprising stretches of alternating positively-charged amino acids, arginine and lysine, such as rkrkLVPRGrkrkLVPRGrkrkLVPRGrkrk (with lower-case letters denoting d-amino acids), that interacts with the negatively charged therapeutic agent, e.g., heparin.

The masking moiety and the therapeutic agent may also interact indirectly. In a specific embodiment, the masking moiety and the therapeutic agent may both be conjugated to a third agent, e.g., a nanoparticle. Examples of nanoparticles that can serve as third agents include, but are not limited to, gold nanoparticles, silica nanoparticles, dextran, albumin, PEG, dendrimers and PLGA particles. The masking moiety, e.g., a peptide, and the therapeutic agent, e.g. bivalirudin, may be conjugated to the third agent, e.g., a nanoparticle, through a number of different chemistries that may comprise, but are not limited to, NHS-amine coupling, maleimide-sulfhydryl coupling and click chemistry.

In one embodiment, the third agent is an iron oxide nanoparticle. Accordingly, in one example, the nanocomplex of the invention comprises the masking moiety, e.g., a peptide, and the therapeutic agent, e.g., bivalirudin, that are both conjugated to a nanoparticle, e.g., an iron oxide nanoparticle.

The “sensor responsive to a stimulus”, as comprised in the masking moiety of the nanocomplexes of the invention, is any molecular entity that is modified in response to a stimulus. For example, the sensor may undergo a conformation change, a cleavage, a binding, or a degradation in response to a stimulus. In one embodiment, the sensor comprises a protease sensitive sequence, e.g., a thrombin cleavage sequence, and is cleaved in response to exposure to the stimulus, e.g., thrombin. For example, the sensor may comprise the thrombin cleavage sequence: P₄-P₃-P₂-P₁-P_{1′}-P_{′} comprising arginine (R) in position P₁. Examples of such thrombin cleavage sequences may include P₄-P₃-P₂-P₁-P_{1′}-P_{2′} comprising arginine (R) in position Pᵢ and glycine (G) in position P₂ and position P₁; or P₄-P₃-P₂-P₁-P_{1′}-P_{2′} comprising hydrophobic residues in position P₄ and position P₃, proline (P) in position P₂, arginine (R) in position P₁, and non-acidic amino-acids in position P_{1′} and position P_{2′}. In a specific embodiment, the sensor comprises the peptide sequence: leucine-valine-proline-arginine-glycine (LVPRG), a well-known thrombin substrate.

In some embodiments, the nanocomplexes of the invention are self-assembling. The self-assembling nanocomplexes are spontaneously formed after their components, e.g., the masking moiety and the therapeutic agent, are mixed together. Formation of the self-assembling nanocomplexes does not require additional manipulations, e.g., chemical reaction or conjugation steps. For example, the nanocomplex comprising a charged masking moiety, e.g., a positively charged peptide, and a charged therapeutic agent, e.g., heparin, is self-assembling.

In some embodiments, the nanocomplexes of the invention are also self-titrating. A self-titrating nanocomplex responds to changes in the strength of a stimulus, thereby modulating the level of the resulting biological activity by the therapeutic agent. For example, in nanocomplexes comprising a peptide as a masking moiety and heparin as the therapeutic agent, the amount of heparin released from the nanocomplex in response to the stimulus, e.g., thrombin, is proportional to the amount of thrombin activity present. Such nanocomplex deploys more anti-coagulant under thrombotic conditions, yet, is more tolerant of healthy coagulation processes (see FIGS. 1B-D).

### III. Pharmaceutical Compositions of the Invention

Although the nanocomplexes of the invention may be used without additional carriers, nanocomplexes of the invention may also be formulated as pharmaceutical compositions further comprising a pharmaceutically acceptable carrier or diluent. As used herein, a “pharmaceutical composition” can be a formulation containing the nanocomplexes, in a form suitable for administration to a subject. Suitable pharmaceutically acceptable carriers may contain inert ingredients which do not inhibit the biological activity of the therapeutic agents contained in the nanocomplex. The pharmaceutically acceptable carriers that may be used in the pharmaceutical composition are any carriers that are biocompatible, i.e., non-toxic, non-inflammatory, non-immunogenic and devoid of other undesired reactions upon the administration to a subject. Standard pharmaceutical formulation techniques can be employed, such as those described in Remington: the Science and Practice of Pharmacy, 19^{th} edition, Mack Publishing Co., Easton, Pa. (1995).

The pharmaceutical composition can be in bulk or in unit dosage form. The unit dosage form can be in any of a variety of forms, including, for example, a capsule, an IV bag, a tablet, a single pump on an aerosol inhaler, or a vial. The quantity of a nanocomplex in a unit dose is the effective amount of the nanocomplex that can vary according to the chosen administration route. A variety of routes are contemplated, including topical, oral, transmucosal or parenteral, including transdermal, subcutaneous, intravenous, intramuscular, intraperitoneal and intranasal. For oral administration, the nanocomplex may be combined with a suitable solid or liquid carrier or diluent to form capsules, tablets, pills, powders, syrups, solutions, suspensions, or the like.

The tablets, pills, capsules, and the like can contain from about 1 to about 99 weight percent of the active ingredient and a binder such as gum tragacanth, acacias, corn starch or gelatin; excipients such as dicalcium phosphate; a disintegrating agent such as corn starch, potato starch or alginic acid; a lubricant such as magnesium stearate; and/or a sweetening agent such as sucrose, lactose or saccharin. When a dosage unit form is a capsule, it may contain, in addition to materials of the above type, a liquid carrier such as a fatty oil.

For parental administration, a nanocomplex may be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. In one embodiment, the pharmaceutical composition is not a hydrogel composition. Under ordinary conditions of storage and use, these preparations contain a preservative to prevent the growth of microorganisms.

In addition to the formulations described above, a formulation can optionally include, or be co-administered with one or more additional drugs, e.g., anti-coagulants, antihypertensives, anti-inflammatories, antibiotics, antifungals, antivirals, immunomodulators, antiprotozoals, steroids, decongestants, bronchodilators, antihistamines, anticancer agents, and the like. The formulation may also contain preserving agents, solubilizing agents, chemical buffers, surfactants, emulsifiers, colorants, odorants and sweeteners.

### IV. Methods for Using the Nanocomplexes of the Invention

The present invention also provides methods for treating or preventing a disease or a condition, e.g., a hypercoagulable state, in a subject. The invention also provides methods for treating a subject in need of anti-thrombotic therapy or prophylaxis. The methods include administering to the subject a therapeutically effective amount or prophylactically effective amount of a nanocomplex of the invention.

As used herein, a “subject” includes either a human or a non-human animal, preferably a vertebrate, and more preferably a mammal. A subject may include a transgenic organism. Most preferably, the subject is a human, such as a human suffering from or predisposed to developing thrombosis or a hypercoagulable state.

A “hypercoagulable state”, or “thrombophilia”, as used herein, refers to any blood clotting disorder that is characterized by excessive coagulation or any other condition associated with excessive coagulation. In one embodiment, the conditions associated with excessive coagulation include, but are not limited to, any condition characterized by an increased risk of myocardial infarction, pulmonary embolism or a stroke, such as hypertension or cardiovascular disease, e.g., coronary occlusion, arteriosclerotic heart disease (ASHD) or coronary thrombosis.

The nanocomplexes of the invention may be administered to a subject using any mode of administration known in the art, including, but not limited to subcutaneous, intravenous, intramuscular, intraocular, intrabronchial, intrapleural, intraperitoneal, intraarterial, lymphatic, cerebrospinal, and any combinations thereof. In one embodiment, the nanocomplexes are administered parenterally, e.g., intravenously. In a further embodiment, the nanocomplexes are administered intravenously by a bolus dose, via continuous infusion, e.g., via an intravenous drip.

The nanocomplexes of the invention may also be administered using a dosing schedule that comprises an initial dose and one or more subsequent maintenance doses. For example, the schedule may include an initial intravenous bolus dose, followed by one or more maintenance doses administered intravenously by continuous infusion, e.g., via an intravenous drip. The exact dosing schedule for a nanocomplex of the invention will depend on the dosing schedule recommended for the specific therapeutic agent comprised in the nanocomplexes.

Other modes of administration include epidural, intracerebral, intracerebroventricular, nasal administration, intraarterial, intracardiac, intraosseous infusion, intrathecal, and intravitreal, and pulmonary. The mode of administration may be appropriately determined by a one of skill in the art, e.g., a physician, in the course of the treatment.

In some embodiments, the nanocomplex is administered to a subject in an amount effective to prevent, reduce or inhibit clot formation in a subject. Such amount includes the amount of the nanocomplex that releases the dose of the therapeutic agent that is effective to treat or prevent a hypercoagulable state, e.g., reduce a risk of a myocardial infarction. Such amount also includes the amount of the nanocomplex that maintains the desired concentration of the therapeutic agent in the blood of a subject that is effective to treat or prevent a hypercoagulable state, e.g., reduce a risk of a myocardial infarction.

An “effective amount”, as used herein, includes the amount of a nanocomplex that, when administered to a subject for treating a condition, e.g., a hypercoagulable state, provides the amount of therapeutic agent that is sufficient to effect treatment of a condition, e.g., a hypercoagulable state, (e.g., by diminishing, ameliorating or maintaining the existing disease or one or more symptoms of disease).

The “effective amount,” as used herein, includes the amount of a nanocomplex that, when administered to a subject who is at risk of developing or may be predisposed to a disease or condition, e.g., a hypercoagulable state, such as a cardiovascular disease, provides the amount of the therapeutic agent that is sufficient to prevent or ameliorate the disease or one or more symptoms of the disease. The “effective amount,” as used herein, also includes the amount of a nanocomplex that, when administered to a subject who is at risk of developing or may be predisposed to a disease or condition, e.g., a hypercoagulable state, such as a cardiovascular disease, is able to maintain the concentration of the therapeutic agent in the blood of the subject that is sufficient to prevent or ameliorate the disease or one or more symptoms of the disease. Ameliorating the disease includes slowing the course of the disease or reducing the severity of later-developing disease.

The “effective amount” may vary depending on the exact nature of the nanocomplex, how the nanocomplex is administered, the rate and the efficiency of release of the therapeutic agent from the nanocomplex, the amount of the therapeutic agent present in the nanocomplex, the disease and its severity and the history, age, weight, family history, genetic makeup, the types of preceding or concomitant treatments, if any, and other individual characteristics of the patient to be treated.

In one embodiment, the contemplated dose range for the nanocomplex comprising heparin is the range that provides the dose of heparin from about 1 U/kg to about 500 U/kg, e.g., about 1 U/kg, about 5 U/kg, about 10 U/kg/ about 15 U/kg, about 20 U/kg, about 25 U/kg, about 30 U/kg, about 35 U/kg, about 40 U/kg, about 45 U/kg, about 50 U/kg, about 55 U/kg, about 60 U/kg, about 65 U/kg, about 70 U/kg, about 75 U/kg, about 80 U/kg, about 85 U/kg, about 90 U/kg, about 95 U/kg, about 100 U/kg, about 110 U/kg, about 120 U/kg, about 130 U/kg, about 140 U/kg, about 150 U/kg, about 160 U/kg, about 170 U/kg, about 180 U/kg, about 190 U/kg, about 200 U/kg, about 210 U/kg, about 220 U/kg, about 230 U/kg, about 240 U/kg, about 250 U/kg, about 250 U/kg, about 260 U/kg, about 270 U/kg, about 280 U/kg, about 290 U/kg, about 300 U/kg, about 310 U/kg, about 320 U/kg, about 330 U/kg, about 340 U/kg, about 350 U/kg, about 360 U/kg, about 370 U/kg, about 380 U/kg, about 390 U/kg, about 400 U/kg, about 410 U/kg, about 420 U/kg, about 430 U/kg, about 440 U/kg, about 450 U/kg, about 460 U/kg, about 470 U/kg, about 480 U/kg, about 490 U/kg, or about 500 U/kg.

In another embodiment, the recommended dose range of the nanocomplex comprising heparin is the range that provides the dose of heparin from about 1 U/kg to about 160 U/kg, e.g., about 1 U/kg, about 5 U/kg, about 10 U/kg, about 15 U/kg, about 20 U/kg, about 25 U/kg, about 30 U/kg, about 35 U/kg, about 40 U/kg, about 45 U/kg, about 50 U/kg, about 55 U/kg, about 60 U/kg, about 65 U/kg, about 70 U/kg, about 75 U/kg, about 80 U/kg, about 85 U/kg, about 90 U/kg, about 95 U/kg, about 100 U/kg, about 110 U/kg, about 120 U/kg, about 130 U/kg, about 140 U/kg, about 150 U/kg, about 160 U/kg. These doses of the nanocomplex may be administered intravenously, e.g., as bolus doses.

The initial administration of the bolus dose of the nanocomplex comprising heparin may be followed by one or more maintenance doses required to maintain therapeutic levels of heparin in the blood of the subject. Maintenance of the therapeutic levels may be accomplished by administering the nanocomplexes of the invention via intravenous infusion, e.g., an intravenous drip. Maintenance doses of the nanocomplexes may range from about 0.1 to about 30 U/kg/hour, e.g., about 0.1 U/kg/hour, about 1 U/kg/hour, about 3 U/kg/hour, about 4 U/kg/hour, about 5 U/kg/hour, about 6 U/kg/hour, about 7 U/kg/hour, about 8 U/kg/hour, about 9 U/kg/hour, about 10 U/kg/hour, about 11 U/kg/hour, about 12 U/kg/hour, about 13 U/kg/hour, about 14 U/kg/hour, about 15 U/kg/hour, about 16 U/kg/hour, about 17 U/kg/hour, about 18 U/kg/hour, about 19 U/kg/hour, about 20 U/kg/hour, about 21 U/kg/hour, about 22 U/kg/hour, about 23 U/kg/hour, about 24 U/kg/hour, about 25 U/kg/hour, about 26 U/kg/hour, about 27 U/kg/hour, about 28 U/kg/hour, about 29 U/kg/hour or about 30 U/kg/hour.

The recommended dose range of the nanocomplex comprising bivalirudin is the range that provides the dose of bivalirudin of from about 0.01 U/kg to about 10 mg/kg, e.g., about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. These doses of the nanocomplex may be administered intravenously, e.g., as bolus doses.

The initial administration of the bolus dose of the nanocomplex comprising bivalirudin may be followed by one or more maintenance doses required to maintain therapeutic levels of bivalirudin in the blood of the subject. The maintenance doses of the nanocomplexes comprising bivalirudin may range from about 0.01 mg/kg/hour to about 10 mg/kg/hour, e.g., about 0.01 U/kg/hour, about 0.02 U/kg/hour, about 0.03 U/kg/hour, about 0.04 U/kg/hour, about 0.05 U/kg/hour, about 0.06 U/kg/hour, about 0.07 U/kg/hour, about 0.08 U/kg/hour, about 0.09 U/kg/hour, about 0.1 U/kg/hour, about 0.2 U/kg/hour, about 0.3 U/kg/hour, about 0.4 U/kg/hour, about 0.5 U/kg/hour, about 0.6 U/kg/hour, about 0.7 U/kg/hour, about 0.8 U/kg/hour, about 0.9 U/kg/hour, about 1 U/kg/hour, about 2 U/kg/hour, about 3 U/kg/hour, about 4 U/kg/hour, about 5 U/kg/hour, about 6 U/kg/hour, about 7 U/kg/hour, about 8 U/kg/hour, about 9 U/kg/hour, or about 10 U/kg/hour. The maintenance doses of the nanocomplexes comprising bivalirudin may be administered for a length of time ranging from about 4 hours to about 20 hours after the initial administration of the bolus dose.

It may be necessary to use doses of nanocomplex outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

In some embodiments, the nanocomplex is administered in combination with other therapeutic agents or other therapeutic regimens. For example, other agents or other therapeutic regimens suitable for treating a hypercoagulable state, e.g., a cardiovascular disease, may include angiotensin converting enzyme inhibitors (ACE inhibitors), angiotension II receptor blockers, antiarrhythmics, antiplatelet drugs, anti-hypertensives, e.g., beta-blockers, calcium channel blocker drugs, anti-coagulants, digoxin, diuretics or nitrates. In one embodiments, the nanocomplex of the invention is administered in combination with aspirin.

The present invention is next described by means of the following examples. However, the use of these and other examples anywhere in the specification is illustrative only, and in no way limits the scope and meaning of the invention or of any exemplified form. The invention is not limited to any particular preferred embodiments described herein. Many modifications and variations of the invention may be apparent to those skilled in the art and can be made without departing from its spirit and scope. The contents of all references, patents and published patent applications cited throughout this application, including the figures, are incorporated herein by reference.

### EXAMPLES

### Example 1

### Synthesis of Charged Peptides

Heparin is a strongly anionic material which may be neutralized in the clinical setting via sequestration by cationic, arginine-rich protamine peptides that bind heparin to form non-reactive nanocomplexes (Rossmann P. et al., Virchows Archiv B Cell Pathol., 1982, 40:81-98). Synthetic peptide antidotes of heparin require a minimum amount of positive charge in order to completely neutralize the functional activity of heparin (DeLucia A. et al., J. of Vascular Surgery, 1993, 18:49-58). Accordingly, a long peptide with multiple cationic regions separated by protease-cleavable sequences was designed. It was expected that this peptide would veil heparin function while intact, but will break down into fragments that would be too small to inhibit heparin activity in response to thrombin-induced cleavage. Sequence of the peptide contained LVPRG, a well-known thrombin substrate separated by stretches of alternating positively-charged amino acids, arginine and lysine, and was as follows: rkrkLVPRGrkrkLVPRGrkrkLVPRGrkrk (lower-case letters denote d-amino acids). Peptides were synthesized by standard FMOC solid-phase peptide synthesis at Koch Institute Biopolymers Core Facility or at Tufts University Core Facility, lyophilized, and resuspended at the concentration of 5 mg/mL in ddH₂O.

A formulation of the peptide-heparin that is free from the instability and aggregation characteristics of charged nanocomplexes in physiological solutions and at high concentrations was then developed. The instability and aggregation can negatively impact in vivo performance by decreasing circulation time and increasing risk of lung entrapment, as evidenced by bare protamine-heparin nanocomplexes that rapidly deposit in locations such as the glomerular basement membrane and do not remain in circulation (Rossmann et al., Virchows Archive B Cell Pathol (1982), 40:81-98). To enhance stability of nanocomplexes in ionic solutions, to prevent non-specific protein interactions, to improve biodistribution and to increase circulation time through steric stabilization, the FDA-approved polymer, poly(ethylene glycol) (PEG), a biocompatible polymer widely used in drug delivery applications, was conjugated to the peptides via amine-ester chemistry. Specifically, 5 kDa PEG was covalently attached to the lysines of the peptides by incubating 5 mg/mL stock peptide solution with amine-reactive 5,000 Da poly(ethylene glycol)-succinimidyl valerate (PEG-SVA, Laysan Bio Inc.) for 1 hour at room temperature.

### Example 2

### Synthesis and Characterization of Heparin-Peptide Nanocomplexes

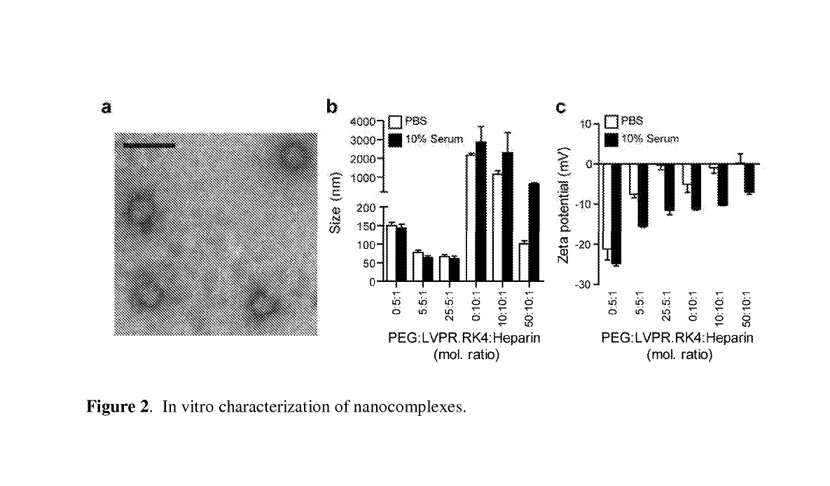

The PEG-conjugated peptides as described in Example 1 were combined with heparin (sodium salt from porcine mucosa, Sigma) at a fixed concentration of 20 U/mL (−0.1 mg/mL) unless reported otherwise. To determine the optimal ratio of PEG:peptide:heparin, nanocomplexes with peptide:heparin ratios between 1:1 to 10:1 and PEG:peptide ratios between 1:1 to 25:1 were formed, incubated in PBS buffer or 10% serum for 1 hour, and their size and zeta potential was measured by dynamic light scattering. For measurements in ionic solutions, 10×PBS stock was added to pre-formed nanocomplex solutions for a final concentration of 1×PBS. For measurements in serum, bovine serum (Gibco) was added to a concentration of 10% (v/v). Mean hydrodynamic diameter was determined via dynamic light scattering of a 50 μL sample at 20 U/mL heparin (ZetaSizer Nano Series, Malvern) and is shown in FIG. 2B. Zeta potential was measured via electrophoretic light scattering on a 900 μL particle sample at 20 U/mL (ZetaSizer Nano Series), and the results are shown in FIG. 2C. The morphology of the nanocomplexes were visualized by negatively staining samples with 2% uranyl acetate and imaging via transmission electron microscopy using a FEI Tecnai Spirit operated at 80 kV. The PEG-peptides and heparin self-assembled into spherical nanoscale complexes, as was confirmed by transmission electron microscopy, as is shown in FIG. 2A.

Increasing the peptide:heparin ratios from 5:1 to 10:1 increased the size of nanocomplexes in PBS and 10% serum by an order of magnitude (from ˜100 nm to 1000 nm), possibly due to complex aggregation (See FIG. 2B). On the other hand, increasing the PEG:peptide ratio appeared to help stabilize the complex size in both PBS and 10% serum (thus decreased the complex size). The zeta potential of nanocomplexes was negative overall, with the addition of PEG bringing the charge closer to neutral (See FIG. 2C). Based on the in vitro data, the ratios that generated the smallest particles with the highest steric stabilization were 5:1 PEG:peptide with 5:1 peptide:heparin, leading to an overall 25:5:1 PEG:peptide:heparin ratio. This ratio was determined to be the best suited for in vivo applications, where the nanocomplex must avoid aggregation and non-specific binding to cells and proteins.

### Example 3

### Cytotoxicity Assays

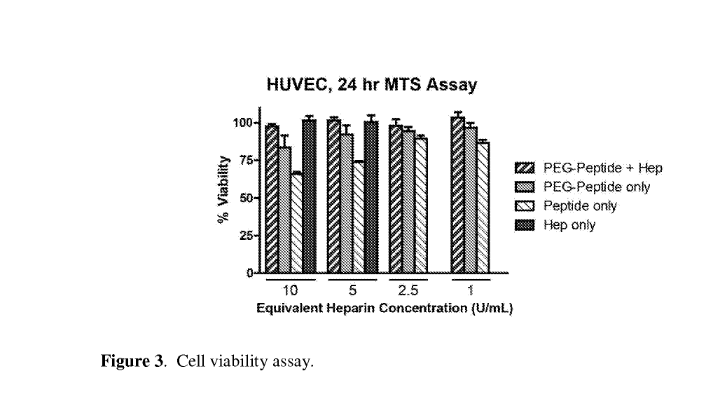

Since cationic peptides may be cytotoxic (Ellerby et al., Nat Med (1999), 5: 1032-1038; Hancock, R. E. W., Lancet (1997), 349(9049):418-422; Wyman et al., Biochemistry (1997), 36(10):3008-3017), the cytotoxicity of nanoparticles at 25:5:1 PEG:peptide:heparin ratio (25:5:1 LVPR.RK4 particles) was determined. For the cytotoxicity assays, Human umbilical vein endothelial cells (HUVEC, Passage 9) were cultured in EGM-2 media (Lonza) on a 96-well plate. When the cells reached 70% confluency, nanocomplexes, free peptide, or free heparin were added as 9×stocks in PBS, diluted in EGM-2. After 24 hours elapsed, cell viability was quantified by an MTS Assay (CellTiter AQueous One, Promega) based on OD490 after 1 hour incubation. No cell toxicity was observed up to 10 U/mL, which corresponds to a ˜1000 U/kg heparin dose in the bloodstream (See FIG. 3). The 25:5:1 LVPR.RK4 particle formulation was used for the remainder of the in vitro and in vivo experimentation.

### Example 4

### Veiling and Unveiling of Heparin Nanoparticles In Vitro

The purpose of this experiment was to show that the nanocomplexes could function to self-regulate the release of heparin in response to clotting activity. The LVPR.RK4 peptides were designed with three thrombin-cleavable stretches to enable release of heparin in response to thrombin activity.

First, to characterize thrombin-triggered disassembly of the nanocomplexes, nanocomplexes formulated as described above using fluorescently labeled heparin (FITC heparin, Polysciences) were incubated for 30 minutes at 37° C. with or without thrombin (500 nM) at 37° C. and were assayed using analytical FPLC. Specifically, analytical samples of nanocomplexes, nanocomplexes incubated with thrombin and of free heparin were applied to a Superdex 200 column pre-equilibrated with PBS. Absorbance of the column effluent was monitored at a wavelength of 488 nm by a UV flow-through detector.

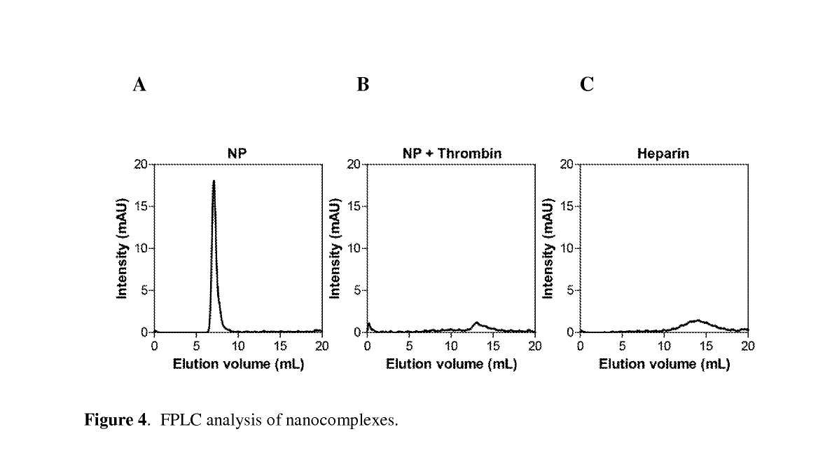

As is shown in FIG. 4, the chromatogram for a sample containing intact nanocomplex exhibited a sharp peak at ˜7mL (panel A). In contrast, in the chromatogram for the sample containing thrombin-treated nanocomplex, this peak is absent and is replaced by a broad peak from ˜11-17 mL, corresponding to the peak observed in the chromatogram for the sample containing free heparin (panels B and C). These results supports the hypothesis that heparin is shielded from external charge when complexed and that thrombin cleavage of the peptide causes dissociation of the complexes and the release of heparin.

To test for full sequestration of heparin, the Azure II assay was used. Azure II is a metachromatic dye that exhibits a shift in absorbance upon electrostatic interactions. Samples containing intact nanocomplexes, nanocomplexes incubated with thrombin or free heparin were mixed with 0.1 mg/ml Azure II solution at a 1:10 volumetric ratio, and the absorbance was read at 530 nm with SpectraMAX Plus spectrophotometer (Molecular Devices).

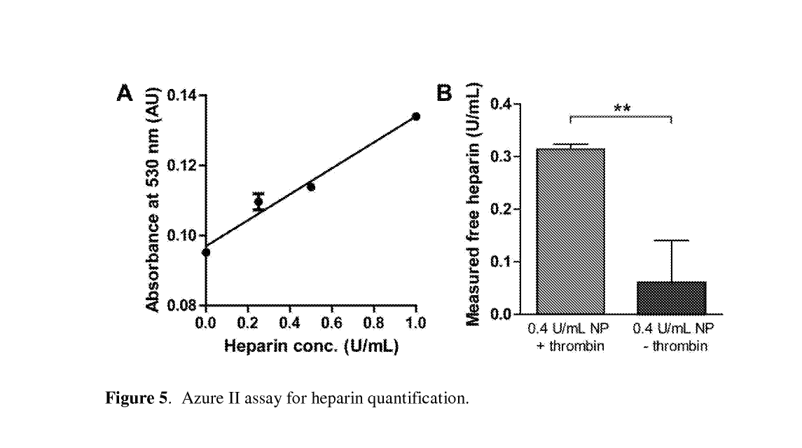

As shown in FIG. 5, intact nanoparticles showed little interaction with Azure II, while nanocomplexes that have been incubated with thrombin caused a dramatic increase in the absorbance at 530 nm. Taken together, this data supports the model that the peptide successfully sequesters the negative charge of heparin when the nanoparticles are intact, while thrombin cleavage of the peptide leads to peptide fragments that are no longer able to neutralize heparin charge, thereby releasing the heparin.

The sequestration of heparin charge by uncleaved peptide and release of heparin charge upon peptide cleavage indicates that the heparin function will be unveiled in response to thrombin activity. The Factor Xa (FXa) assay (anti-FXa assay) was used to determine the functional state of the heparin in samples containing intact and thrombin-cleaved nanocomplexes. This assay uses a chromogenic substrate to measure the FXa enzyme activity of the sample and to determine the amount of heparin in the sample, which is inversely proportional to anti-coagulant levels. The anti-FXa assay (Sekisui Diagnostics) was performed according to the manufacturer's instructions. The release of heparin was determined by incubating nanocomplexes with various concentrations of human thrombin (Haemotologic Technologies) at 37° C. for the various amounts of time. The activity of the released heparin was then determined using the anti-FXa assay. In the anti-FXa assay, a test sample or heparin standard is added to a fixed amount of antithrombin III (ATIII). A fixed amount of factor Xa is then added to the sample, resulting in the formation of inactive ATIII-Xa complexes. Residual Xa is then measured using either a clotting-based assay or chromogenic assay. The residual Xa activity is inversely proportional to the heparin concentration in the sample and can be quantitated from a calibration curve.

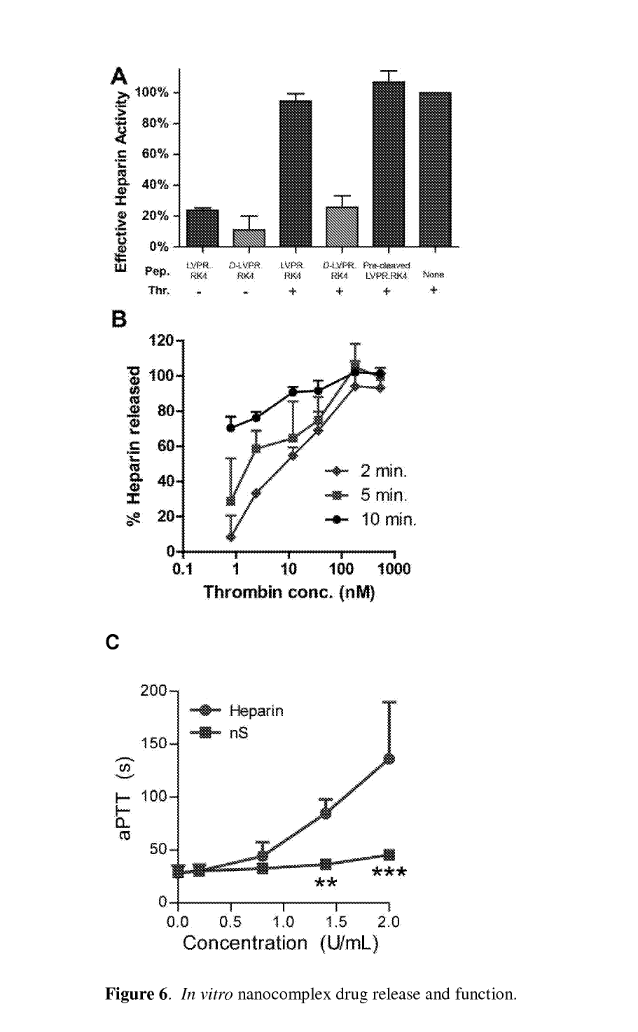

As is shown in FIG. 6A, intact nanocomplexes demonstrated ˜80% reduced heparin activity relative to free heparin, while heparin activity was fully restored in complexes that were fully cleaved by thrombin. Incubation of thrombin with nanocomplexes formed with all-D-amino acid LVPR.RK4 that are unrecognizable by endogenous proteases led to minimal unveiling of heparin activity. This further supports the idea that the release of anti-coagulant is directly driven by thrombin. As a final control, heparin incubated with peptide that had already been pre-cleaved with thrombin was assayed. These samples exhibited full heparin activity, confirming that cleaved fragments lose their ability to inhibit heparin activity. These results show that the peptide-heparin nanocomplexes veil heparin activity in intact form and release fully functional heparin in response to thrombin cleavage.

Anti-FXa assay was also used to determining the kinetics of heparin release when the nanocomplexes are incubated with varying concentrations of thrombin over time. In whole blood or plasma, thrombin concentrations as low as ˜1 nM are required to initiate the subsequent burst in thrombin generation (up to 100-500 nM) that is needed to produce a stable fibrin clot (Brummel et al., Blood (2002), 100:148-152; Orfeo et al., PLOS One (2011), 6(11):e27852). Over-anticoagulation may delay or dampen this thrombin generation cycle and disrupt fibrin formation, resulting in prolonged bleeding, whereas insufficient anticoagulation may fail to inhibit the unchecked coagulation activity that leads to thrombosis. Therefore, the purpose of this experiment was to probe the response of nanocomplexes to dynamic thrombin levels.

As is shown in FIG. 6B, % heparin released from the nanocomplexes increased with the increasing thrombin concentration, reaching a plateau of nearly complete release (>95%) in response to 180 nM thrombin for all incubation times tested (2 minutes, 5 minutes and 10 minutes). Similarly, heparin release increased over time in response to thrombin exposure across the range of all concentrations tested. These data indicate that heparin release is a function of thrombin activity and time, behavior which supports the self-titrating negative feedback design of the nanocomplex. These findings also support the idea that the release of heparin payload by the nanocomplexes occurs at physiologically relevant thrombin concentrations.

### Example 5

### Veiled Nanocomplexes Do Not Prolong Clotting Time In Vitro

The effect of the nanocomplexes on a physiologic coagulation pathway was tested using a standard activated partial thromboplastin time (aPTT) clotting assay, which is also used to monitor heparin levels of patients. This assay measures the amount of time needed for a plasma sample to form a clot in vitro when the contact pathway of coagulation is triggered by addition of an exogenous aPTT reagent. For the assay, varying concentrations of nanocomplexes or free heparin were added to 50 μl of APTT reagent (Thermo Scientific) at 37° C. for 3 minutes. Subsequently, 50 μl of 25 mM CaCl₂ (Sigma) pre-incubated at 37° C. were added to samples and clotting was monitored via absorbance at 605 nm with a SpectraMAX Plus spectrophotometer (Molecular Dynamics).

As can be seen in FIG. 6C, in human plasma spiked with nanocomplexes, the clotting time increased modestly in a dose dependent manner from ˜29 s in the absence of nanocomplexes, up to 46 s (˜1.5x of control) for 2 U/mL. In contrast, plasma samples spiked with heparin exhibited substantial lengthening of observed clotting times, reaching ˜44 s (˜1.5× of control) with exposure to only 0.4 U/mL, and reaching as long as ˜136 s (˜5× of control) for 2.0 U/mL. Prolonged aPTT that stems from heparin anticoagulation may indicate an increased risk of potentially fatal bleeding (Eikelboom, J. W., Hirsh, J., Thromb Haemost (2006), 96(5):547-52).

Current clinical guidelines for aPTT monitoring of heparin dosing recommend dose ranges that yield an aPTT approximately ˜1.5-2.3× of the control bleeding time (Eikelboom, J. W., Hirsh, J., Thromb Haemost (2006), 96(5):547-52), affording a relatively narrow therapeutic range. Moreover, even these guidelines are non-standardized, necessitating that each laboratory establish their own reference and therapeutic ranges, which further complicates the management of drug administration. In human plasma samples spiked with free heparin, the clotting time increased dramatically in a dose-dependent manner from ˜29 s in the absence of heparin (0 U/mL) up to ˜44s (˜1.5 fold over control) with exposure to only 0.8 U/mL, and as long as ˜136 s (˜5 fold over control) for 2.0 U/mL (FIG. 6C). In contrast, plasma samples spiked with identical doses of heparin nanocomplexes exhibited significantly shorter increases in clotting time, reaching 46 s (˜1.5 fold over control) only at a dose of 2 U/mL (P<0.001 by Student's t-test, n=3-4 per condition). These results indicate that the nanocomplex formulation largely veils heparin's intrinsic effect on aPTT, indicating that the need for aPTT monitoring may not be necessary when heparin is given in this context. Furthermore, given that the aPTT is not prolonged beyond the clinical recommendation (>2.3× of control) even at high equivalent doses of heparin, the nanocomplexes may yield a much wider therapeutic window with less risk of overdose than observed with traditional heparin treatment. These results also indicate that relative to the free form, an equivalent dose of heparin sequestered within nanocomplexes leads to a significantly shorter aPTT, which is a marker where longer clotting times are clinically associated with higher risk of bleeding (Granger, C. B., et al.,Circulation (1996), 93:870-78; Anand, S. S., et al., Circulation (2003),107:2884-88). This demonstrates that nanocomplexes offer a wider therapeutic window with a reduced risk of bleeding when compared to traditional heparin (UFH) treatment.

### Example 6

### Nanocomplexes Remain Veiled in the Absence of Thrombosis and Do Not Increase Bleeding

In the bloodstream, the nanocomplexes must face a complex milieu of proteins and plasma components without systemic release of their cargo until exposed to sites of thrombus formation. To demonstrate that the anti-coagulant property of the nanocomplexes remains suppressed in vivo under healthy conditions, two circulation time experiments were performed. In the first experiment, mice were injected with nanocomplexes containing fluorescently labeled heparin or free heparin (100 U/kg, n=3), and their blood was sampled over time by measuring the amount of fluorescence in the plasma. Specifically, mice (n=3 per condition) were injected via tail vein with nanocomplexes or free heparin formulated with 1×PBS. Blood samples collected thorough retro-orbital blood draw and centrifuged at 2,900×g for 5 minutes to isolate plasma. The plasma was then analyzed by fluorimetry using the Spectramax Gemini EM Fluorescence Microplate Reader (Molecular Devices) at excitation/emission wavelengths of 485/538 nm.

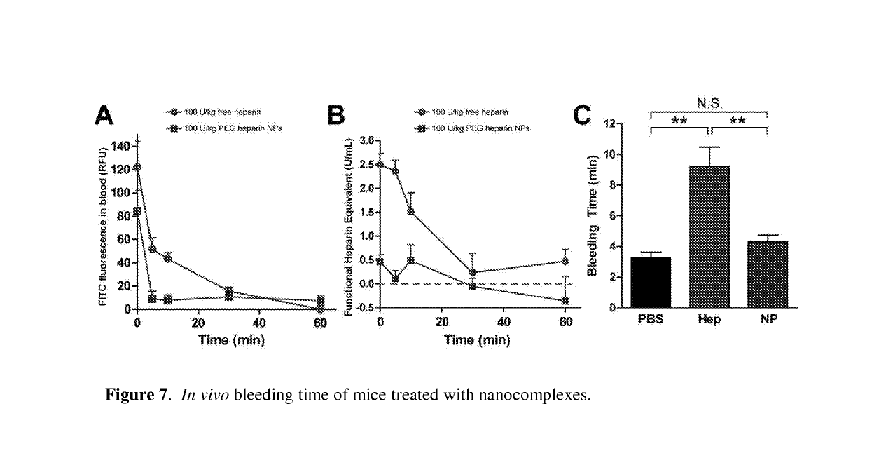

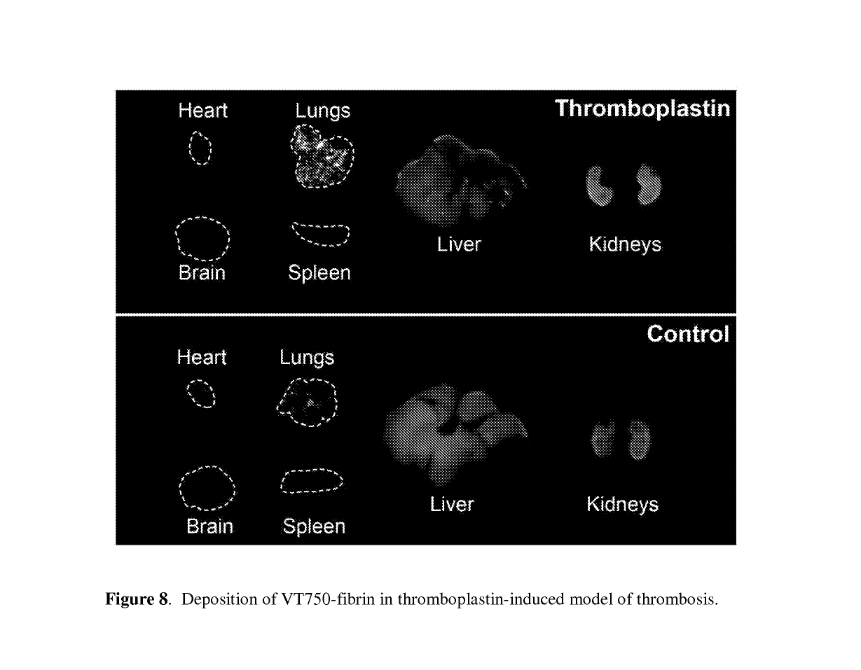

The results, as presented in FIG. 7A, indicate that the nanocomplexes exhibit a rapid initial clearance half-life within minutes, and that a small fraction of the injected dose may remain in circulation for greater than an hour.

In the second experiment, mice were again injected with nanocomplexes or free heparin and, and their plasma was collected and evaluated for functional heparin activity. Specifically, healthy female Swiss Webster mice (3-4 months, n=3 per condition) were injected via tail vein with LVPR.RK4 nanocomplexes or free heparin at 100 U/kg, formulated in isotonic 5% dextrose solution in water (D5W). Blood samples were collected in tubes containing 3.2% sodium citrate (Sigma) for a final volume ratio of 9:1 (blood:citrate) through retro-orbital blood draws and centrifuged at 2,900×g for 5 minutes to isolate the plasma. Heparin activity was then measured using the anti-FXa assay.

The results, as presented in FIG. 7B, demonstrate that the nanocomplexes successfully veil heparin function in vivo, leading to reduced levels of heparin activity in the blood pool relative to free heparin.

The purpose of the next experiment was to test if designing the nanocomplexes to release more heparin in response pathological levels of thrombin activity would temper its impact on bleeding time during hemostatic events. Moderate to potentially fatal bleeding is the primary side effect of nearly all clinical anti-coagulants, including heparin, and hemostasis is largely driven by platelet activation and plug formation, a process requiring significantly lower thrombin concentrations than needed for fibrin clot formation (Dawson et al., J. of Biol. Chem. (1994), 269:15989-92).

To assess the effect of nanocomplex administration on bleeding tail transection on mice was performed five minutes after they were treated intravenously with nanocomplexes. Specifically, mice (n=5-7 per condition) were anesthetized with isofluorane gas and administered nanocomplexes (200 U/kg, n=5), heparin (200 U/kg, n=7), or PBS control (n=5). After 5 minutes, 2 mm of distal mouse tail was removed by scalpel. Bleeding time was determined by lightly dabbing the tail with kimwipe tissues (Kimtech) until bleeding fully ceased for at least 1 minute. All experimental protocols involving animals were approved by the MIT Committee on Animal Care.

As is shown in FIG. 7C, the mean bleeding time of nanocomplex-treated mice (˜4.3 min, bar graph labeled “NP”) was only 30% longer than that of normal PBS-treated mice (˜3.3 min, bar graph labeled “PBS”). In contrast, free heparin increased the mean bleeding time of mice (˜9.2 min, bar graph labeled “Hep”) by greater than 280% over the mean control bleeding time. The reduction in bleeding time of nanocomplexes-treated mice versus free heparin-treated mice is indicative that there was insufficient thrombin activation at the injury site to release enough heparin to lead to a significant impact on hemostasis-mediated clotting. Furthermore, the sequestration of heparin in complex form may also block free heparin-platelet interactions that contribute to prolongation of bleeding time (Gao et al., Blood (2011), 117(18):4946-4952). Taken together, these data show that the nanocomplexes remain in circulation while keeping heparin in a veiled state and reduce the protracted bleeding time associated with heparin administration.

### Example 7

### Nanocomplexes Prevent Thrombosis In Vivo

To evaluate the ability of nanocomplexes to prevent thrombosis in vivo, a thromboplastin-induced model of pulmonary thromboembolism was utilized. As is demonstrated in FIG. 8, this model is characterized by deposition of microembolisms primarily in the lungs (Weiss et al., Blood (2002), 100(9):3240-3244; Smyth et al., Blood (2001), 98(4):1055-1062). For the pulmonary embolism assay, bovine fibrinogen (Sigma) was reacted with near-infrared fluorochromes (Vivotag-750-NHS, Perkin-Elmer) at a 2:1 fluorophore:protein molar ratio in PBS for 1 hour and purified by column centrifugation (100 kDa cutoff, Millipore) to remove unreacted fluorophores. Ampules of thromboplastin from rabbit brain (Sigma, #44213) were reconstituted with 2 mL of PBS each.

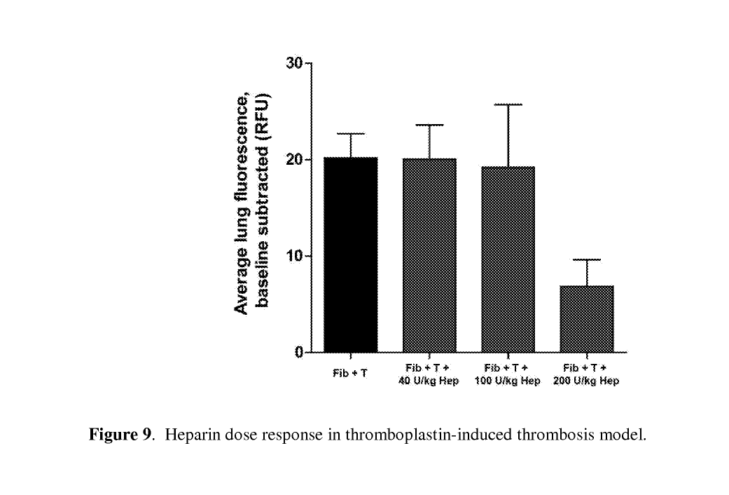

To ascertain the initial dosing ranges for the nanocomplexes, mice were dosed with thromboplastin and escalating doses of free heparin (40 U/kg, 100 U/kg and 200 U/kg heparin). Deposition of VT750-fibrin in the lungs of the mice was quantified using ex vivo infrared imaging. The results, presented in FIG. 9, demonstrate that free heparin, when administered at the doses of 40 U/kg or 100 U/kg, did not significantly reduce the amount of VT750-fibrin deposition, while free heparin, when administered at the dose of 200 U/kg, reduced VT750-fibrin deposition by more than two-fold. Accordingly, the dose of nanocomplexes corresponding to heparin doses of 200 U/kg was chosen for subsequent experiments.

Mice (n=5 per condition) were anesthetized with isofluorane gas and co-administered nanocomplexes (200 U/kg), free heparin (200 U/kg), or PBS control and 1 nmol of VT750-fibrinogen via tail vein injection. After 5 minutes, mice were injected with thromboplastin (2 uL/g). After 30 minutes, mice were euthanized with CO₂ and the lungs were harvested and imaged on the LI-COR Odyssey Infrared Imaging System. Fibrin deposition was then quantified using ImageJ software. For histologic analysis, paraffin-embedded sections of lungs were prepared (Koch Institute Histology Core). Lungs were first fixed by incubating in 4% paraformaldehyde overnight. Hematoxylin and eosin immunochemical staining of lung sections was used to visualize clots in the lungs.

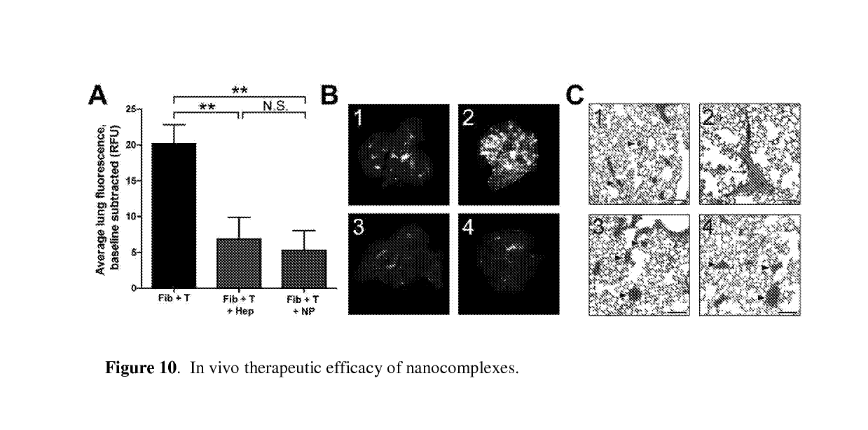

The results of the in vivo experiments are shown in FIG. 10. Specifically, FIG. 10A shows average fibrin deposition in the lungs of mice dosed with thromboplastin (T) and nanocomplexes or free heparin (n=5 per condition, p<0.01); FIG. 10B shows ex vivo fluorescent imaging of VT750-fibrin in lungs of mice administered (1) PBS, (2) thromboplastin, (3) thromboplastin+heparin, and (4) thromboplastin+nanocomplexes; and FIG. 10C shows hematoxylin and eosin immunochemical staining of lungs of mice treated as described for FIG. 10B.

As is shown in FIGS. 10A and 10B, the lungs of mice treated with nanocomplexes showed a 75% reduction (p<0.01, ANOVA) in fibrin deposition compared to the lungs of control-treated mice. The reduction in fibrin deposition caused by the nanocomplexes (bar graph labeled “Fib+T+NP”) was equivalent to the effect demonstrated by the corresponding dosage of free heparin (graph labeled “Fib+T+Hep”). Moreover, histological analysis showed that microvessels in the lungs of control-treated animals contain thrombi (arrows in FIG. 10C), whereas such vessels in animals treated with either free heparin or nanocomplexes were largely patent, i.e., unblocked with functional blood flow, as was evidenced by the presence of red blood cells (arrow heads in FIG. 10C). These results, combined with the results from the tail bleeding assay, demonstrate that the nanocomplexes prevent thrombosis as effectively as a matching dose of free heparin, but with a significantly reduced risk of bleeding.

### Example 8

### Ex Vivo Characterization of Nanocomplexes in Human Plasma

To further analyze the anticoagulant mechanism of the nanocomplexes, we performed real-time thrombin generation assays in human plasma. For this assay, varying concentrations of nanocomplexes or free heparin were added to 20 μL of control normal human plasma (Thermo Scientific). Real time thrombin generation was measured using the Technothrombin TGA kit (Technoclone) using the RD reagent according to manufacturer instructions. The fluorescence was monitored using a TECAN Infinite M200 Pro and thrombin generation was calculated using the corresponding Excel evaluation software provided by Technoclone.

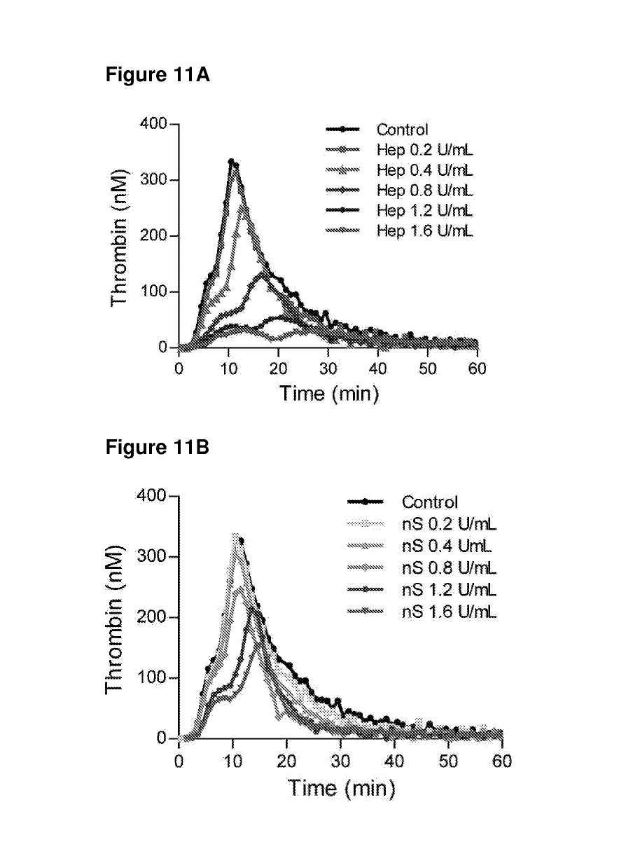

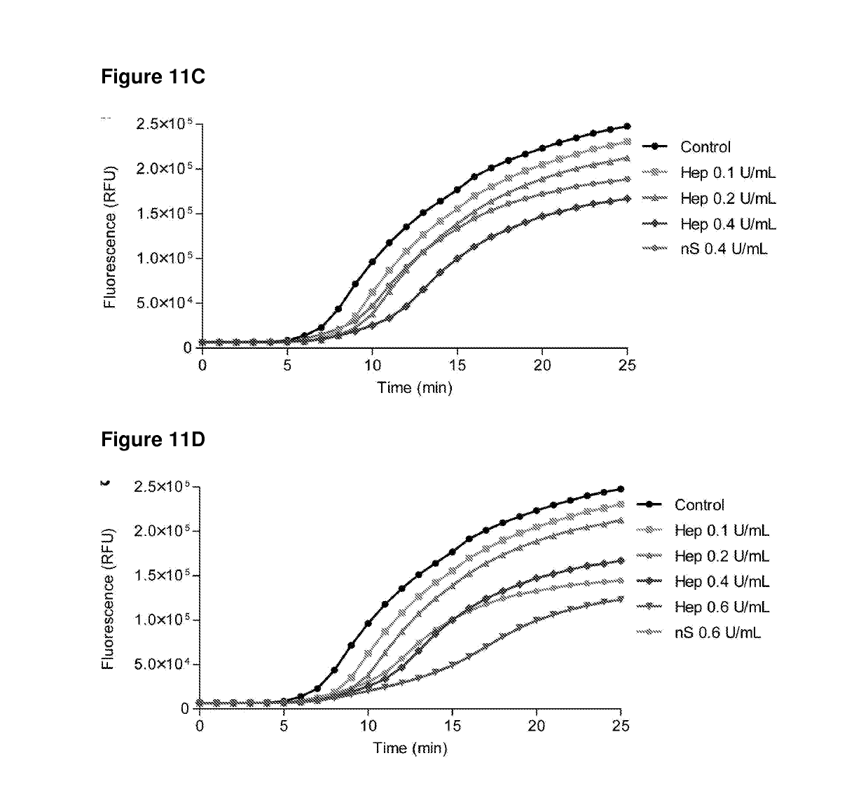

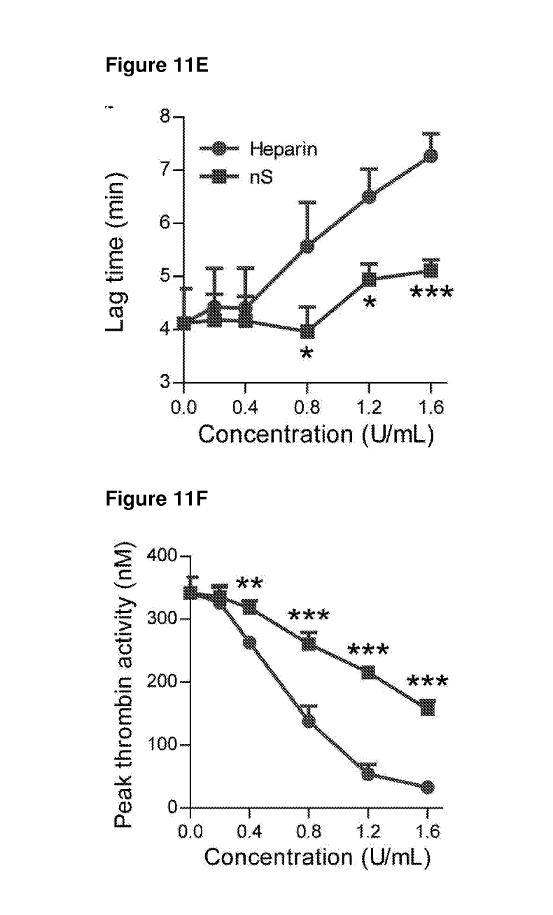

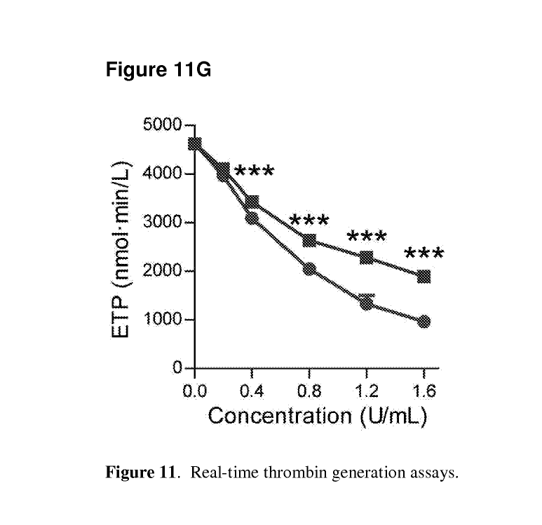

As can be seen in FIG. 11, the thrombin generation curves of free heparin and nanocomplexes exhibited distinct patterns (FIG. 11A, FIG. 11B, FIG. 11C, and FIG. 11D. The time to initial thrombin formation (lag time), which correlates with clot initiation, was prolonged in a dose-dependent manner by free heparin as expected. In contrast, the lag time of plasma samples with identical doses of nanocomplexes did not change markedly as the effective concentration of heparin increased (FIG. 11E), P<0.01 by Student's t-test, n=3 per condition) (Hemker, H. C., et al., Pathophysiol. Haemost. Thromb. (2002), 32:249-53; Hemker, H. C., et al.,Pathophysiol. Haemost. Thromb. (2003), 33:4-15). These results were consistent with the aPTT assay (see Example 5 and FIG. 6C), and support the model that nanocomplexes initially veil complexed heparin and block its activity. Following the onset of clotting, nanocomplexes demonstrated anticoagulant activity similar to free heparin by reducing the maximum (peak) and total (endogenous thrombin potential, ETP) thrombin generation relative to the untreated control (0 U/mL), demonstrating that the nanocomplexes release active heparin during the clotting process. However, the magnitude of the reductions in peak thrombin and ETP was significantly smaller for the nanocomplexes at equivalent heparin concentrations greater than 0.2 U/mL (P<0.001 by Student's t-test, n=3 per condition; FIG. 11F and 11G). This trend was particularly apparent in the peak thrombin activity, which shows that the generation of activated thrombin is needed to first release heparin from the nanocomplexes in order to block further coagulation, as opposed to free heparin that is active from the outset. The ETP of the nanocomplexes never decreased below 40% of the baseline level even at the highest dose tested (1.6 U/mL), which was over twice the maximum recommended dose of unfractionated heparin (UFH) (Eikelboom, J. W. and Hirsh, J. Thromb. Haemost. (2006), 96:547-52; Hirsh, J. et al., Chest (2001), 119:64S-94S; Raschke, R. A., et al., Ann. Intern. Med. (1993) 119:874-81), whereas the equivalent concentration of free heparin reduced the ETP by ˜80%. This observation demonstrates that the nanocomplexes of the invention are able to buffer against safety risks from dose escalation of heparin, as previous studies have shown that reductions in ETP greater than 80% relative to normal baseline have been correlated with risk of bleeding in patients (Hemker, H. C., et al., Thromb. Haemost. (2006), 96:553-61; Duchemin, J. Thromb. Haemost. (2008), 99:767-73). This is an improved and unexpected property of the nanocomplexes of the present invention.

1. A stimulus responsive nanocomplex, comprising:
a therapeutic agent; and
a masking moiety comprising a sensor responsive to a stimulus, wherein the masking moiety prevents the therapeutic agent from exerting its biological activity, and wherein said sensor is modified in the presence of said stimulus, thereby allowing said therapeutic agent to exert its biological activity.

2. The stimulus responsive nanocomplex of claim 1, wherein said masking moiety comprises a proteinaceous compound.

3. (canceled)

4. The stimulus responsive nanocomplex of claim 2, wherein said sensor comprises a protease sensitive sequence.

5. (canceled)

6. The stimulus responsive nanocomplex of claim 4, wherein said stimulus is an agent capable of cleaving said protease sensitive sequence.

7.-9. (canceled)

10. The stimulus responsive nanocomplex of claim 1, wherein the therapeutic agent is a blood homeostasis agent.

11.-12. (canceled)

13. The stimulus responsive nanocomplex of claim 1, wherein said nanocomplex is self-assembling.

14. The stimulus responsive nanocomplex of claim 1, wherein said nanocomplex is self-titrating.

15. The stimulus responsive nanocomplex of claim 13, wherein said therapeutic agent and said masking moiety interact directly with each other to form the nanocomplex.

16. The stimulus responsive nanocomplex of claim 15, wherein said therapeutic agent is a charged therapeutic agent, and wherein said masking moiety is a charged moiety.

17.-34. (canceled)

35. The stimulus responsive nanocomplex of claim 2, wherein said proteinaceous compound is further conjugated to a polymeric agent.

36. The stimulus responsive nanocomplex of claim 35, wherein said polymeric agent is polyethylene glycol (PEG).

37. A stimulus responsive nanocomplex, comprising:
a charged therapeutic agent; and
a charged peptide comprising a sensor responsive to a stimulus, wherein the charge of the peptide is opposite to the charge of the charged therapeutic agent thereby allowing the formation of the nanocomplex and wherein in the presence of said stimulus said sensor is modified thereby releasing the charged therapeutic agent from the nanocomplex.

38. The stimulus responsive nanocomplex of claim 37, wherein said charged therapeutic agent is negatively charged and wherein said charged peptide is positively charged.

39. (canceled)

40. The stimulus responsive nanocomplex of claim 37, wherein said charged therapeutic agent is a blood homeostasis agent.

41.-42. (canceled)

43. The stimulus responsive nanocomplex of claim 37, wherein said sensor is a protease sensitive sequence.

44.-48. (canceled)

49. The stimulus responsive nanocomplex of claim 37, wherein said charged peptide masks the biological activity of said charged therapeutic agent.

50. A nanocomplex for the treatment or prevention of thrombosis, said nanocomplex comprising:
a negatively charged anti-thrombotic agent; and
a positively charged peptide, wherein said peptide comprises a protease sensitive sequence.

51. (canceled)

52. The nanocomplex of claim 50, wherein said peptide is in excess of said anti-thrombotic agent within said nanocomplex.

53. The nanocomplex of claim 50, wherein said nanocomplex is formed by the self-assembly of said anti-thrombotic agent and said peptide.

54.-60. (canceled)

61. A method of treating a subject in need thereof, the method comprising administering to said subject an effective amount of the nanocomplex of claim 1, thereby treating said subject.

62.-65. (canceled)
